(19) United States
(12) Patent Application Publication
(10) Pub. No.: US 2025/0049596 A1
(43) Pub. Date: Feb. 13, 2025

(54) NON-VASCULAR DRUG-ELUTING STENT

(71) Applicant: Gareth Morris-Stiff, Cleveland Heights, OH (US)

(72) Inventor: Gareth Morris-Stiff, Cleveland Heights, OH (US)

(21) Appl. No.: 18/797,117

(22) Filed: Aug. 7, 2024

Related U.S. Application Data
(60) Provisional application No. 63/531,519, filed on Aug. 8, 2023.

(51) Int. Cl. A61F 2/90

(52) U.S. Cl. CPC A61F 2/90; A61F 2230/0006; A61F 2230/0069; A61F 2240/001; A61F 2250/0067

(57) ABSTRACT

A non-vascular drug-eluding stent is provided that includes a self-expanding metal stent (SEMS) body having an outside annular surface, and a continuous polymeric layer that is loaded with a chemotherapeutic agent. The polymeric layer surrounds at least part of the outside annular surface of the SEMS body, and the polymeric layer includes SIBS. Other aspects related to methods of making the non-vascular drug-eluding stent and treatment using the non-vascular drug-eluding stent are also described and claimed.

### CROSS-REFERENCE TO RELATED APPLICATION(S)

The present disclosure claims priority from U.S. Provisional Appl. No. 63/531,519, filed on Aug. 8, 2023, entitled “NON-VASCULAR DRUG-ELUTING STENT,” herein incorporated by reference in its entirety.

### BACKGROUND

### 1. Field

The present disclosure relates to stents for treating diseased or restricted lumens or ducts of the gastrointestinal tract.

### 2. State of the Art

Stents, such as non-vascular stents, are often used to open or maintain patency of constricted lumens or to provide drainage through obstructed lumens of non-vascular tubular organs or tissue. Such lumens can become constricted or obstructed as a results of injury or disease. For example, bile ducts may be obstructed as a result of cancer of the pancreas or the common bile duct, thereby causing an excessive accumulation of bilirubin in the body. A biliary stent may be placed in the bile duct to allow the bile to drain through the bile duct into the small intestine. The biliary stent can employ a thin silicon liner that extends along the interior annular surface of the stent to prevent tissue ingrowth into the stent lumen. The silicon liner is inert and plays no therapeutic role.

Cancer of the gastrointestinal tract, such as cancer of the pancreas or cancer of the common bile duct, is typically treated by systemic administration of a chemotherapeutic agent to kill cancer cells. Some of the chemotherapeutic agents commonly used include gemcitabine, 5-fluorouracil, irinotecan, oxaliplatin, paclitaxel, capecitabine, cisplatin, and docetaxel.

### SUMMARY

A non-vascular drug-eluding stent is provided that includes a self-expanding metal stent (SEMS) body having an outside annular surface, and a continuous polymeric layer that is loaded with a chemotherapeutic agent. The polymeric layer surrounds at least part of the outside annular surface of the SEMS body, and the polymeric layer includes SIBS.

In embodiments, the chemotherapeutic agent can be or include paclitaxel. In other embodiments, the chemotherapeutic agent can be or include gemcitabine, 5-fluorouracil, irinotecan, oxaliplatin, capecitabine, cisplatin, docetaxel, another suitable agent, or combinations thereof.

In embodiments, the polymeric layer can be configured to release or elute the chemotherapeutic agent over time for localized delivery of the chemotherapeutic agent to tissue at or near a non-vascular implant site of the stent to kill cancer cells at or near the implant site of the stent over time.

In embodiments, the polymeric layer can be configured to have elasticity such that the polymeric layer expands and contracts radially with the radial expansion and contraction of the SEMS body during use.

In embodiments, the non-vascular drug-eluding stent can be configured to be deployed in a non-vascular implant site such as, for example, the gastrointestinal tract such as the bile duct, esophagus, pancreas, duodenum, stomach, or colon.

Other aspects related to methods of making the non-vascular drug-eluding stent and treatment using the non-vascular drug-eluding stent are also described and claimed.

### BRIEF DESCRIPTION OF THE DRAWINGS

For a more complete understanding of the disclosed subject matter, and advantages thereof, reference is now made to the following descriptions taken in conjunction with the accompanying drawings as summarized below.

FIG. 1A is a schematic perspective diagram of a non-vascular drug-eluting stent according to the present disclosure.

FIG. 1B is a schematic perspective diagram of the SEMS body of the stent of FIG. 1A.

FIG. 1C is a schematic cross-section diagram of the stent of FIG. 1A along the section line labeled 1C/1D.

FIG. 1D is a schematic cross-section diagram of an alternate embodiment of the stent of FIG. 1A along the section line labeled 1C/1D.

FIG. 2 is a schematic diagram of a non-vascular drug-eluting stent according to the present disclosure that is deployed within a bile duct to treat a cancerous tumor adjacent the bile duct.

### DETAILED DESCRIPTION OF THE PREFERRED EMBODIMENTS

The present disclosure provides a non-vascular stent that is configured to be placed within a diseased or restricted lumen or duct of the gastrointestinal tract, such as at diseased site within the biliary duct. The stent includes a self-expanding metal stent (SEMS) body whose outside annular surface (or part thereof) is surrounded by a continuous polymeric layer that is loaded with a chemotherapeutic agent. In embodiments, the chemotherapeutic agent can be or include paclitaxel. In other embodiments, the chemotherapeutic agent can be or include gemcitabine, 5-fluorouracil, irinotecan, oxaliplatin, capecitabine, cisplatin, docetaxel, another suitable agent, or combinations thereof. In embodiments, the polymeric layer of the stent includes SIBS. SIBS is a non-biodegradable polymer having a triblock polymer backbone including polystyrene-polyisobutylene-polystyrene. Polyisobutylene (PIB) is a soft elastomeric material with a Shore hardness of approximately 10A to 30A. Polyisobutylene is copolymerized with polystyrene to form SIBS. The hardness of SIBS can have a range from soft (Shore 10A) to hard (Shore 100D) depending on the relative amounts of polyisobutylene and styrene used to form the SIBS. In this manner, SIBS can be adapted to have the desired hardness and elastomeric qualities. Details of SIBS is set forth in U.S. Pat. Nos. 5,741,331; 6,102,939; 6,197,240; 6,545,097, which are hereby incorporated by reference in their entirety. The SIBS of the continuous polymeric layer of the stent can be loaded with the chemotherapeutic agent such that the chemotherapeutic agent releases or elutes from the polymeric layer over time for localized delivery of the chemotherapeutic agent to tissue at or near the implant site of the stent to kill cancer cells at or near the implant site of the stent over time.

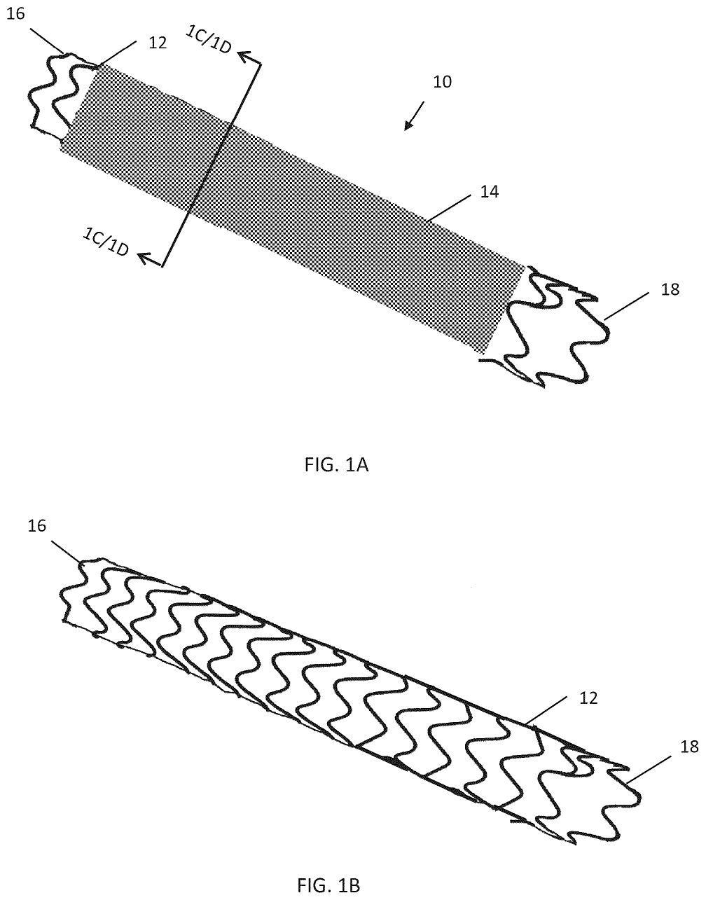

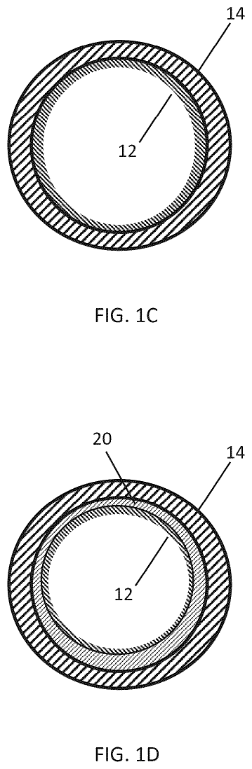

Referring to FIG. 1A, a stent 10 according to the present disclosure has a hollow tubular shape defined by SEMS body 12. FIG. 1B shows the SEMS body 12 alone. The outside annular surface (or part thereof) of the SEMS body 12 is surrounded by a continuous polymeric layer 14 that is loaded with a chemotherapeutic agent. In embodiments, the chemotherapeutic agent can be or include paclitaxel. In other embodiments, the chemotherapeutic agent can be or include gemcitabine, 5-fluorouracil, irinotecan, oxaliplatin, capecitabine, cisplatin, docetaxel, another suitable agent, or combinations thereof. The openings defined by the SEMS body 12 can be occluded by the polymeric layer 14. The polymeric layer 14 can be configured to have elasticity such that the polymeric layer 14 expands and contracts radially with the radial expansion and contraction of the SEMS body 12 during use. Specifically, the SEMS body 12 and the polymeric layer 14 can be configured to radially contract with one another when one or both ends of the SEMS body 12 is (are) pulled away from SEMS body 12 to apply tenson to the device in an axial direction. This contraction can occur when loading the stent 10 into its delivery system or possibly when removing the stent 10 from the implant site. The SEMS body 12 and the polymeric layer 14 can also be configured to radially expand with one another automatically under the radial expansion forces imparted by the SEMS body 12 when deployed from its delivery system. In FIG. 1A, the polymeric layer 14 is depicted schematically as being opaque and thus hiding the underlying SEBS body 12. In practice, the polymeric layer 14 need not be opaque and thus the underlying SEBS body 12 can be visible through polymeric layer 14.

Although a feature of the present disclosure is that the polymeric layer 14 surrounds the outside annular surface (or part thereof) of the SEMS body 12, other surfaces of the SEMS body 12 may be covered by the polymeric layer 14. For example, the inside annular surface of the SEMS body 12 may be covered by the polymeric layer 14 to provide even greater surface area for the release of the chemotherapeutic agent. Furthermore, the polymeric layer 14 need not surround or cover the proximal end 16 of the SEMS body 12 or the distal end 18 of the SEMS body 12 as shown in FIG. 1A to improve stent anchoring and reduce stent migration. In other embodiments, the polymeric layer 14 can surround and cover the proximal end 16 of the SEMS body 12, the distal end 18 of the SEMS body 12 or both.

In embodiments, the proximal end 16 of the SEMS body 12, the distal end 18 of the SEMS body 12, or both can incorporate a retrieval loop (not shown). When tension is applied to the retrieval loop using a surgical tool (such as forceps), the tension can cause the entire length and diameter of the stent 10 to narrow to help facilitate removal of the stent.

In embodiments, the SEMS body 12 can be formed from a shape memory metal (such as nitinol). The SEMS body 12 can include cross-hatched, braided or interconnected rows of metal that are assembled into a tube-like structure as shown in FIG. 1B. In embodiments, the SEMS body 12 can have the shape of a coil stent, spiral stent, zigzag stent, or a mesh stent (including a patterned stent such as a braided, woven, or knitted stent). The polymeric layer 14 loaded with the chemotherapeutic agent is configured to surround or cover the SEMS body 12 or portion thereof. The stent 10 can be configured in a contracted state within a wire-guided delivery system. The wire-guided delivery system can be introduced into a diseased or restricted lumen or duct of the gastrointestinal tract, such as at a diseased site within the biliary duct. The stent 10 can be deployed at the site by withdrawing the outer catheter of the delivery system as is conventional. The stent 10 can be configured to self-expand radially under the radial expansion forces imparted by the SEMS body 12 when deployed from the delivery system. The self-expanding mechanism of the stent 10 is configured to fix and retain the stent 10 within the diseased or restricted lumen or duct, such as at the diseased site within the biliary duct.

In embodiments, the stent 10 according to the present disclosure can be sized and configured to be deployed in any non-vascular implant site such as, for example, the gastrointestinal tract such as the bile duct, esophagus, pancreas, duodenum, stomach, or colon. The size of the stent can depend on its specific application.

In embodiments, in order to form the polymeric layer 14 of sufficient thickness that surrounds and covers the outer surface of the SEMS body 12, a viscous mixture can be formed from a SIBS polymer, a chemotherapeutic agent, and a suitable solvent. The mixture can be applied to the SEMS body 12 or portion thereof as a coating and then the solvent can be evaporated from the structure. This step can be repeated until the polymeric layer 14 of desirable thickness has been obtained.

In other embodiments, the chemotherapeutic agent can be combined with SIBS by adding the chemotherapeutic agent to a SIBS melt during thermoplastic processing. The combined SIBS and the chemotherapeutic agent (in melt phase) can then be used to coat the outside annular surface (or part thereof) of the SEMS body 12 to form the continuous polymeric layer 14 surrounding the outside annular surface (or part thereof) of the SEMS body 12.

In yet other embodiments, the chemotherapeutic agent can be added to SIBS after forming the continuous polymeric layer surrounding the outside annular surface (or part thereof) of the SEMS body 12. As an example, the chemotherapeutic agent can be dissolved in a solvent that is compatible with both SIBS and the chemotherapeutic agent. A continuous polymeric layer of SIBS can be formed on the external annular surface (or part thereof) of the SEMS body. Subsequently, the solution can be contacted with the continuous polymeric layer of SIBS such that the therapeutic agent is loaded (e.g., by leaching/diffusion) into the SIBS. For this purpose, the stent or stent portion can be immersed or dipped into the solution, or the solution can be applied to the stent or stent portion, for example, by spraying, printing dip coating, immersing in a fluidized bed and so forth. The stent or stent portion can subsequently be dried, with the chemotherapeutic agent remaining therein.

In still another embodiment, the chemotherapeutic agent may be provided within a matrix comprising SIBS that forms the continuous polymeric layer 14 on the outside annular surface (or part thereof) of the SEMS body 12. The chemotherapeutic agent can also be covalently bonded, hydrogen bonded, or electrostatically bound to the SIBS. As specific examples, nitric oxide releasing functional groups such as S-nitroso-thiols can be provided in connection with SIBS, or SIBS can be provided with charged functional groups that attach to oppositely charged functional groups of the chemotherapeutic agent.

In yet another alternative embodiment, the chemotherapeutic agent can be precipitated onto a continuous SIBS layer that is formed on or surrounds the outside annular surface (or part thereof) of the SEMS body 12. This layer can be subsequently covered with a coating of SIBS (with or without additional chemotherapeutic agent) as described above.

In still another embodiment, an annular-shaped polymeric film comprising SIBS loaded with the chemotherapeutic agent can be formed separately from the SEMS body 12 (for example, by casting or molding techniques) and then rolled or otherwise placed in position surrounding the outside annular surface (or part thereof) of the SEMS body 12.

Hence, when it is stated herein that the SIBS is “loaded” with the chemotherapeutic agent, it is meant that the chemotherapeutic agent is associated with the SIBS polymer in a fashion like those discussed above or in a related fashion.

In some instances a binder may be useful for adhesion to the outside annular surface (or part thereof) of the SEMS body 12. Examples of materials appropriate for binders in connection with the present disclosure include silanes, titanates, isocyanates, carboxyls, amides, amines, acrylates hydroxyls, and epoxides, including specific polymers such as EVA, polyisobutylene, natural rubbers, polyurethanes, siloxane coupling agents, ethylene and propylene oxides.

It also may be useful to add to the stent or portion thereof (which may or may not contain the chemotherapeutic agent) one or more additional polymeric layers (which may or may not contain the chemotherapeutic agent). Such additional polymeric layer(s) may be disposed adjacent to the drug-eluting polymeric layer 14 and serve, for example, as a boundary layer to block or retard diffusion of the chemotherapeutic agent and prevent a burst phenomenon whereby much of the agent is released immediately upon exposure of the stent portion to the implant site. The material constituting the additional polymeric layer(s) may or may not be a SIBS polymer. For example, in one embodiment, one or more intermediate polymeric layers or films 20 can be disposed between the outside surface of the SEMS body 12 and the polymeric layer 14 of the device as shown in FIG. 1D. The intermediate polymeric layer(s) 20 can occlude the openings defined by the SEMS body 12 and act as a substrate that interfaces to the polymeric layer 14. The intermediate polymeric layer(s) 20 can block or retard diffusion of the chemotherapeutic agent into the interior passageway of the stent during use when the stent is implanted in vivo at the implant site. In yet other embodiments, one or more polymeric layers or films can be disposed on the inside surface of the SEMS body 12. Such inside polymeric layer(s) 20 can occlude the openings defined by the SEMS body 12 and act as a substrate that interfaces to the polymeric layer 14. The inside polymeric layer(s) can block or retard diffusion of the chemotherapeutic agent into the interior passageway of the stent during use when the stent is implanted in vivo at the implant site.

It is also possible to form the polymeric layer 14 of the SEMS body 12 with blends by adding one or more other polymers to a SIBS polymer. Examples include the following:
blends can be formed with homopolymers that are miscible with one of the phases of the SIBS copolymer. For example, polyphenylene oxide is miscible with the styrene blocks of polystyrene-polyisobutylene-polystyrene copolymer. This should increase the strength of the polymeric layer or portion thereof.
blends can be made with added polymers or other copolymers that are not completely miscible with the blocks of the SIBS copolymer. The added polymer or copolymer may be advantageous, for example, in that it may alter the release rate of the chemotherapeutic agent from the SIBS polymer of the polymeric layer or portion thereof.
blends can be made with a component such as sugar that can be leached from the polymeric layer of the stent, rendering the stent more porous and controlling the release rate through the porous structure.

The release rate of the chemotherapeutic agent from the therapeutic-agent-loaded SIBS polymer of layer 14 can be varied in a number of ways. Examples include:
varying the molecular weight of the SIBS;
varying the specific constituents selected for the elastomeric and thermoplastic portions of the SIBS and the relative amounts of these constituents;
varying the type and relative amounts of solvents used in processing the SIBS;
varying the porosity of the SIBS;
providing a boundary layer over the SIBS; and
blending the SIBS with other polymers or copolymers.

In embodiments, the stent of the present disclosure can be used to treat cancer in the gastrointestinal tract, such as at diseased site within the biliary duct. The chemotherapeutic agent paclitaxel has for decades been used with great success to treat narrowed segments of the coronary arteries and has been applied to vascular stents where paclitaxel is embedded within a SIBS polymer. However, the stent of the present disclosure are covered with a continuous polymeric SIBS layer loaded with a chemotherapeutic agent (such as paclitaxel) in contrast to the conformal coating process used in vascular stents where openings defined by the stent are not occluded by a covering as such a covering would diminish the vascular tissue uptake of required nutrients from the blood supply. Furthermore, paclitaxel is approved and is a treatment of choice for cancers of both the pancreas and bile duct and so would be an ideal agent to be applied to the outside surface of a SEMS body. It is anticipated that the use of a paclitaxel eluting SEMS would be compared within the context of a trial with the existing SEMS, with both groups in addition receiving standard of care systemic chemotherapy exploring the hypothesis that the addition of local therapy enhances outcome.

It is expected that safety and efficacy of the stent devices as described herein can be assessed in a mini pig models of pancreatic and biliary cancer. These animals are large enough to allow endoscopy to be performed and stents to be deployed.

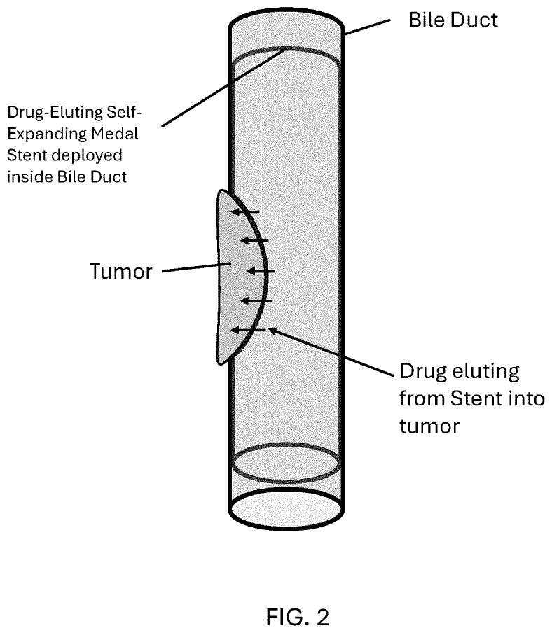

FIG. 2 is a schematic diagram of a non-vascular drug-eluting stent according to the present disclosure that is deployed within a bile duct to treat a cancerous tumor adjacent the bile duct. The stent incorporates polymeric material that is loaded with a chemotherapeutic agent or drug as described herein. The chemotherapeutic agent or drug eludes from the polymeric material over time to treat the cancerous tumor by killing cells and/or inhibiting cell growth of the cancerous tumor. and/or inhibiting cell growth. The self-expanding mechanism of the stent is configured to fix and retain the stent within the bile duct.

In other embodiments, the drug-eluting stent as described herein can be sized and configured to be deployed in another non-vascular implant site, such as, for example, the esophagus, pancreas, duodenum, stomach, or colon of the gastrointestinal tract to treat a cancerous tumor at or near the non-vascular implant site. The size of the stent can depend on its specific application.

The foregoing description has been set forth merely to illustrate the invention and is not intended as being limiting. Each of the disclosed aspects and embodiments of the present invention may be considered individually or in combination with other aspects, embodiments, and variations of the invention. Modifications of the disclosed embodiments incorporating the spirit and substance of the invention may occur to persons skilled in the art and such modifications are within the scope of the present invention. Furthermore, all references cited herein are incorporated by reference in their entirety.

I claim:

1. A non-vascular drug-eluding stent, comprising:
a self-expanding metal stent (SEMS) body having an outside annular surface; and
a continuous polymeric layer that is loaded with a chemotherapeutic agent, wherein the polymeric layer surrounds at least part of the outside annular surface of the SEMS body, and wherein the polymeric layer comprises SIBS.

2. A non-vascular drug-eluding stent according to claim 1, wherein:
the chemotherapeutic agent comprises paclitaxel.

3. A non-vascular drug-eluding stent according to claim 1, wherein:
the chemotherapeutic agent comprises gemcitabine, 5-fluorouracil, irinotecan, oxaliplatin, capecitabine, cisplatin, docetaxel, another suitable agent, or combinations thereof.

4. A non-vascular drug-eluding stent according to claim 1, wherein:
the polymeric layer is configured to release or elute the chemotherapeutic agent over time for localized delivery of the chemotherapeutic agent to tissue at or near a non-vascular implant site of the stent to kill cancer cells at or near the non-vascular implant site of the stent over time.

5. A non-vascular drug-eluding stent according to claim 1, wherein:
the polymeric layer is configured to have elasticity such that the polymeric layer expands and contracts radially with the radial expansion and contraction of the SEMS body during use.

6. A non-vascular drug-eluding stent according to claim 1, which is sized and configured for deployment in a non-vascular implant site.

7. A non-vascular drug-eluding stent according to claim 6, wherein:
the non-vascular implant site is part the gastrointestinal tract, such as a bile duct, esophagus, pancreas, duodenum, stomach, or colon.

8. A method of making a non-vascular drug-eluding stent, comprising:
surrounding at least part of an outer annular surface of a self-expanding metal stent (SEMS) body with a continuous polymeric layer that is loaded with a chemotherapeutic agent, wherein the continuous polymeric layer comprises SIBS.

9. A method according to claim 8, wherein:
the chemotherapeutic agent comprises paclitaxel.

10. A method according to claim 8, wherein:
the chemotherapeutic agent comprises gemcitabine, 5-fluorouracil, irinotecan, oxaliplatin, capecitabine, cisplatin, docetaxel, another suitable agent, or combinations thereof.

11. A method according to claim 8, wherein:
the polymeric layer is configured to release or elute the chemotherapeutic agent over time for localized delivery of the chemotherapeutic agent to tissue at or near a non-vascular implant site of the stent to kill cancer cells at or near the non-vascular implant site of the stent over time.

12. A method according to claim 8, wherein:
the polymeric layer is configured to have elasticity such that the polymeric layer expands and contracts radially with the radial expansion and contraction of the SEMS body during use.

13. A method according to claim 8, wherein:
the non-vascular drug-eluding stent is sized and configured for deployment in a non-vascular implant site.

14. A method according to claim 13, wherein:
the non-vascular implant site is within the gastrointestinal tract, such as the bile duct, esophagus, pancreas, duodenum, stomach, or colon.

15. A method according to claim 8, wherein:
the polymeric layer is applied to the SEMS body by i) forming a viscous mixture from a SIBS polymer, a chemotherapeutic agent, and a suitable solvent, ii) applying the mixture to the SEMS body or portion thereof as a coating, and iii) evaporating the solvent, and optionally repeating this process until the polymeric layer of desirable thickness has been obtained.

16. A method according to claim 8, wherein:
the polymeric layer is applied to the SEMS body by i) combining the chemotherapeutic agent with SIBS during thermoplastic processing, and ii) using the combined SIBS and chemotherapeutic agent in a melt phase to coat the outside annular surface (or part thereof) of the SEMS body.

17. A method according to claim 8, wherein:
the polymeric layer is applied to the SEMS body by adding the chemotherapeutic agent to SIBS after forming a continuous polymeric layer surrounding the outside annular surface (or part thereof) of the SEMS body.

18. A method according to claim 8, wherein:
the chemotherapeutic agent is provided within a matrix comprising SIBS that forms the polymeric layer.

19. A method according to claim 8, wherein:
the chemotherapeutic agent is precipitated onto a continuous SIBS layer that is formed on or surrounds the outside annular surface (or part thereof) of the SEMS body, and the resultant layer is subsequently covered with a coating of SIBS.

20. A method according to claim 8, wherein:
the polymeric layer is applied to the SEMS body by i) forming the an annular-shaped polymeric film comprising SIBS loaded with the chemotherapeutic agent separately from the SEMS body, and ii) placing the polymeric film in position surrounding the outside annular surface (or part thereof) of the SEMS body.

21. A method of treating a non-vascular implant site comprising:
providing a non-vascular stent according to claim 1;
implanting the non-vascular stent at the non-vascular implant site; and
allowing the chemotherapeutic agent to be released from the non-vascular stent over time to treat the non-vascular implant site over time.

22. A method according to claim 21, wherein:
the chemotherapeutic agent comprises paclitaxel.

23. A method according to claim 21, wherein:
the chemotherapeutic agent comprises gemcitabine, 5-fluorouracil, irinotecan, oxaliplatin, capecitabine, cisplatin, docetaxel, another suitable agent, or combinations thereof.

24. A method according to claim 21, wherein:
the polymeric layer is configured to release or elute the chemotherapeutic agent over time for localized delivery of the chemotherapeutic agent to tissue at or near the non-vascular implant site of the stent to kill cancer cells at or near the non-vascular implant site of the stent over time.

25. A method according to claim 23, wherein:
the non-vascular implant site is within the gastrointestinal tract, such as the bile duct, esophagus, pancreas, duodenum, stomach, or colon.
